(19) United States
(12) Patent Application Publication
(10) Pub. No.: US 2015/0088459 A1
(43) Pub. Date: Mar. 26, 2015

(54) MONITORING AND PRESENTING UNSYNCHRONIZED PHYSIOLOGICAL DATA STREAMS

(71) Applicant: COVIDIEN LP, Mansfield, MA (US)

(72) Inventors: BRIAN KEITH RUSSELL, ANNAPOLIS, MD (US); JONATHAN JAMES WOODWARD, ANNAPOLIS, MD (US); DIETRICH OTTO RUEHLMANN, GAITHERSBURG, MD (US); MARJORIE JONES OLSEN, CHARLOTTE, NC (US)

(21) Appl. No.: 14/495,255

(22) Filed: Sep. 24, 2014

Related U.S. Application Data
(60) Provisional application No. 61/882,262, filed on Sep. 25, 2013.

(51) Int. Cl. G06F 19/00; A61B 5/145; A61B 5/0205

(52) U.S. Cl. CPC G06F 19/36; A61B 5/0205; A61B 5/14542; A61B 5/14532; A61B 5/021
USPC 702/176

(57) ABSTRACT

Methods, apparatuses and systems are described for outputting unsynchronized physiological data. The methods may include receiving a plurality of data streams, each data stream representing a physiological parameter for a person. The methods may also include selecting an epoch. Once the epoch is selected, the methods may also include determining, for each of the plurality of data streams, an epochal or summary value to represent each of the physiological parameters for the person in the epoch. The methods may additionally include outputting the epochal values with an indication of the selected epoch.

### CROSS-REFERENCE TO RELATED APPLICATIONS

This application claims priority to U.S. Provisional Patent Application No. 61/882,262, filed on Sep. 25, 2013, the entirety of which is incorporated by reference herein.

### BACKGROUND

The present disclosure relates generally to physiological monitoring systems, and more particularly to presenting data collected through physiological monitoring systems.

Physiological data may be received from multiple sources at different, irregular, and/or unpredictable intervals. For example, a patient may be monitored by sensors that independently measure physiological parameters such as the patient's vital signs. A healthcare provider may also manually enter physiological data that acquired by observing the patient. As such, physiological data may be obtained, reported, and/or recorded from many different sources at many different time intervals. These streams of data, therefore, may not be synchronized.

One way that collected physiological data may be presented to a clinician is in a tabular format. The resulting table may include columns that are each associated with a different physiological parameter (e.g., heart rate, blood pressure, etc.). The resulting table may also include rows that index the collected physiological data by time stamp. When data streams are not synchronized, however, some rows may have one or more empty cells because the physiological parameters associated with the empty cells were not measured during that particular time-stamped time. As a result, the tables produced by these known methods may be unwieldy and difficult to understand.

In some situations, a physiological parameter such as a heart rate may be measured and reported relatively frequently (e.g., every 10 seconds), while another physiological parameter, such as weight, may be monitored and reported relatively infrequently (e.g., once a day). As a result, when the collected data are presented in a tabular format, there may be multiple rows with a cell containing heart rate data and only one row with a cell containing weight observations, thereby making weight observations difficult to locate in the table and/or making it difficult to detect changes and/or trends in weight observations.

In order to create and/or evaluate an overall picture of the patient's current physiological condition, however, it may be beneficial to have an overview of data received from disparate data sources, even when the data is unsynchronized.

### SUMMARY

Because various physiological parameters of a patient may be collected at different times and frequencies, it may be beneficial to a clinician to present such unsynchronized data in a way that is physiologically relevant to the clinician, as well as a way that is simple and useful in identifying physiological trends. In particular, the clinician may benefit from seeing a single most physiologically relevant value for each of a collection of measured vital signs for a given period of time. One method of accomplishing this includes receiving data streams each representing a measured physiological parameter. For each of the received data streams, a most physiologically relevant value is determined for a selected epoch or period of time. The most physiologically relevant value may be the most recently measured value or it may be an average or median value, for example. Once the most physiologically relevant value is determined for each physiological parameter represented by the received data streams, the single most physiologically relevant value for each physiological parameter is output such that a clinician may view a meaningful and simple snapshot of the patient's vital sign measurements.

Once the epoch is selected, there may be a determination, for each of the data streams, of an epochal or summary value to represent each of the physiological parameters for the person in the epoch. The epochal values may be output with an indication of the selected epoch. In one example, the output may be tabular, with the epochal or summary values each being output on a single row corresponding to the selected epoch. Thus, a clinician may view physiologically relevant values for each measured parameter by observing a single row for each epoch, thus avoiding the need to view and evaluate many rows of unsynchronized data that may not be physiologically relevant.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Further scope of the applicability of the described methods and apparatuses will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

A further understanding of the nature and advantages of the present invention may be realized by reference to the following drawings. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 is a block diagram of an example of a physiological parameter monitoring system in accordance with various embodiments;

FIG. 2 is a graphical representation of a table summarizing unsynchronized physiological data by epoch in accordance with various embodiments;

FIG. 3 is a block diagram of an example of an apparatus in accordance with various embodiments;

FIG. 4 is a block diagram of an example of an apparatus in accordance with various embodiments;

FIG. 5 is a block diagram of an example of a server for summarizing unsynchronized physiological data in accordance with various embodiments; and

FIGS. 6 and 7 are flowcharts of various methods for outputting unsynchronized physiological data, in accordance with various embodiments.

### DETAILED DESCRIPTION

In order to efficiently understand the physiological condition of a person, clinicians regularly monitor a plurality of physiological parameters of the person. These parameters may include, for example, the person's heart rate, blood pressure, oxygen saturation levels, glucose levels, weight, etc. The different physiological parameters, however, may be measured at different times and frequencies. Thus, for example, a person's weight may only be recorded once a day while the person's blood pressure may only be recorded a few times per hour and the person's heart rate may be recorded almost continuously. Presenting these unsynchronized data streams to a clinician, therefore, includes a challenge of presenting the most physiologically relevant data for any given period of time.

For example, the clinician may desire to view a snapshot of the person's vital signs for a given period of time. The clinician may benefit from seeing a single most physiologically relevant value for each of the measured vital signs for the given period of time. The recorded physiological data may, however, include multiple values of a single parameter during the given period of time or may include no values of a parameter during the given period of time. The present disclosure includes a method and system for determining and presenting to the clinician the most physiologically relevant values of each collected parameter during a given period of time.

The recorded physiological data may be collected manually or through a physiological monitoring system. One example of a physiological monitoring system is a remote physiological monitoring system. Examples below describe such a system, though it should be understood that any type of physiological monitoring system may provide unsynchronized data streams from which the most physiologically relevant parameter values may be selected for display to a clinician.

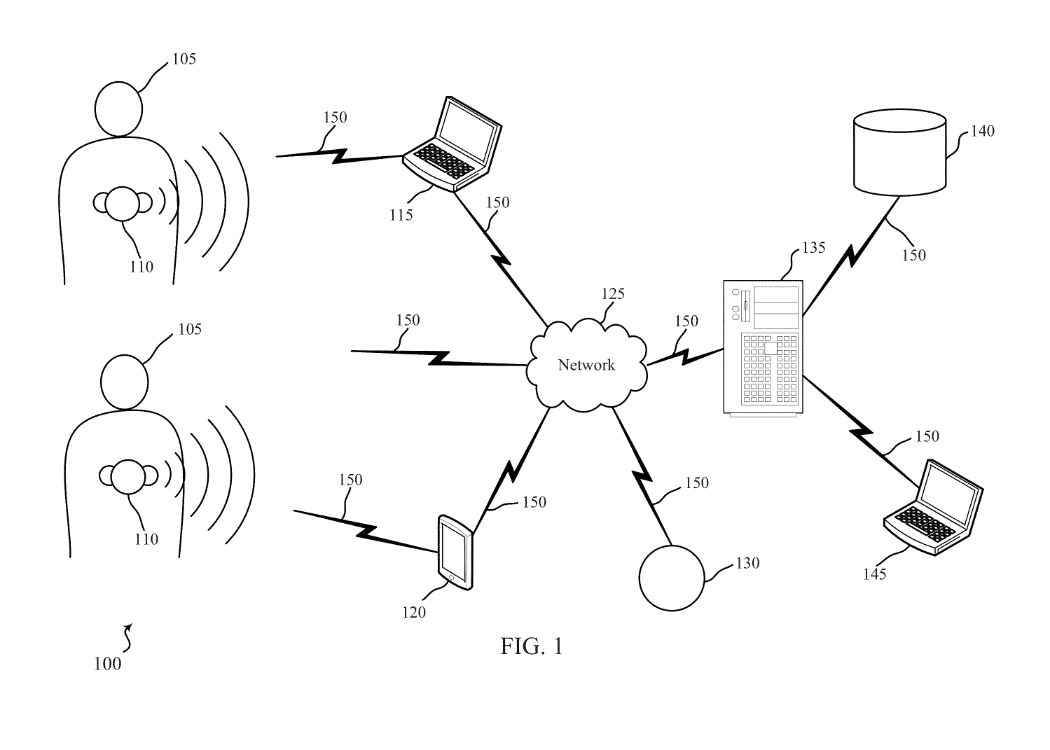

Referring first to FIG. 1, a diagram illustrates an example of a remote physiological parameter monitoring system 100. The system 100 includes persons 105, each wearing a sensor unit 110. The sensor units 110 transmit signals via wireless communication links 150. The transmitted signals may be transmitted to local computing devices 115, 120. Local computer device 115 may be a local care-giver's station, for example. Local computer device 120 may be a mobile device, for example. The local computing devices 115, 120 may be in communication with a server 135 via network 125. The sensor units 110 may also communicate directly with the server 135 via the network 125. Additional, third-party sensors 130 may also communicate directly with the server 135 via the network 125. The server 135 may be in further communication with a remote computer device 145, thus allowing a care-giver to remotely monitor the persons 105. The server 135 may also be in communication with various medical databases 140 where the collected data may be stored.

The sensor units 110 are described in greater detail below. Each sensor unit 110, however, is capable of sensing multiple physiological parameters. Thus, the sensor units 110 may each include multiple sensors such as heart rate and ECG sensors, respiratory rate sensors, and accelerometers. For example, a first sensor in a sensor unit 110 may be an oxygen saturation monitor or a glucose level monitor operable to detect a user's blood oxygen or sugar levels. A second sensor within a sensor unit 110 may be operable to detect a second physiological parameter. For example, the second sensor may be a heart rate monitor, an electrocardiogram (ECG) sensing module, a breathing rate sensing module, and/or any other suitable module for monitoring any suitable physiological parameter. Multiple sensor units 110 may be used on a single person. The data collected by the sensor units 110 may be wirelessly conveyed to either the local computer devices 115, 120 or to the remote computer device 145 (via the network 125 and server 135). Data transmission may occur via, for example, frequencies appropriate for a personal area network (such as Bluetooth or IR communications) or local or wide area network frequencies such as radio frequencies specified by the IEEE 802.15.4 standard.

Each data point recorded by the sensor units 110 may include an indication of the time the measurement was made (referred to herein as a “time stamp”). In some embodiments, the sensor units 110 are sensors configured to conduct periodic automatic measurements of one or more physiological parameters. A person may wear or otherwise be attached to one or more sensor units 110 so that the sensor units 110 may measure, record, and/or report physiological data associated with the patient.

The sensor units 110 may be discrete sensors, each having independent clocks. As a result, sensor units 110 may generate data with different frequencies. The data streams generated by the sensor units 110 may also be offset from each other. The sensor units 110 may each generate a data point at any suitable time interval.

The local computer devices 115, 120 may enable the person 105 and/or a local care-giver to monitor the collected physiological data. For example, the local computer devices 115, 120 may be operable to present data collected from sensor units 110 in a human-readable format. For example, the received data may be output as a display on a computer or a mobile device. The local computer devices 115, 120 may include a processor that may be operable to present data received from the sensor units 110 in a visual format. The local computer devices 115, 120 may also output data in an audible format using, for example, a speaker.

The local computer devices 115, 120 may be custom computing entities configured to interact with the sensor units 110. In some embodiments, the local computer devices 115, 120 and the sensor units 110 may be portions of a single sensing unit operable to sense and display physiological parameters. In another embodiment, the local computer devices 115, 120 may be general purpose computing entities such as a personal computing device, such as a desktop computer, a laptop computer, a netbook, a tablet personal computer (PC), an iPod®, an iPad®, a smart phone (e.g., an iPhone®, an Android® phone, a Blackberry®, a Windows® phone, etc.), a mobile phone, a personal digital assistant (PDA), and/or any other suitable device operable to send and receive signals, store and retrieve data, and/or execute modules.

The local computer devices 115, 120 may include memory, a processor, an output, a data input and a communication module. The processor may be a general purpose processor, a Field Programmable Gate Array (FPGA), an Application Specific Integrated Circuit (ASIC), a Digital Signal Processor (DSP), and/or the like. The processor may be configured to retrieve data from and/or write data to the memory. The memory may be, for example, a random access memory (RAM), a memory buffer, a hard drive, a database, an erasable programmable read only memory (EPROM), an electrically erasable programmable read only memory (EEPROM), a read only memory (ROM), a flash memory, a hard disk, a floppy disk, cloud storage, and/or so forth. In some embodiments, the local computer devices 115, 120 may include one or more hardware-based modules (e.g., DSP, FPGA, ASIC) and/or software-based modules (e.g., a module of computer code stored at the memory and executed at the processor, a set of processor-readable instructions that may be stored at the memory and executed at the processor) associated with executing an application, such as, for example, receiving and displaying data from sensor units 110.

The data input module of the local computer devices 115, 120 may be used to manually input measured physiological data instead of or in addition to receiving data from the sensor units 110. For example, a user of the local computer device 115, 120 may make an observation as to one or more physiological conditions of a patient and record the observation using the data input module. A user may be, for example, a nurse, a doctor, and/or any other medical healthcare professional authorized to record patient observations, the patient, and/or any other suitable person. For instance, the user may measure the patient's body temperature (e.g., using a stand-alone thermometer) and enter the measurement into the data input module. In some embodiments, the data input module may be operable to allow the user to select “body temperature” and input the observed temperature into the data input module, e.g., using a keyboard. The data input module may time stamp the observation (or measurement) with the time the observation is input into the local computer devices 115, 120, or the local computer devices 115, 120 may prompt the user to input the time the observation (or measurement) was made so that the time provided by the user is used to time stamp the data point.

The processor of the local computer devices 115, 120 may be operated to control operation of the output of the local computer devices 115, 120. The output may be a television, a liquid crystal display (LCD) monitor, a cathode ray tube (CRT) monitor, speaker, tactile output device, and/or the like. In some embodiments, the output may be an integral component of the local computer devices 115, 120. Similarly stated, the output may be directly coupled to the processor. For example, the output may be the integral display of a tablet and/or smart phone. In some embodiments, an output module may include, for example, a High Definition Multimedia Interface™ (HDMI) connector, a Video Graphics Array (VGA) connector, a Universal Serial Bus™ (USB) connector, a tip, ring, sleeve (TRS) connector, and/or any other suitable connector operable to couple the local computer devices 115, 120 to the output.

As described in additional detail herein, at least one of the sensor units 110 may be operable to transmit physiological data to the local computer devices 115, 120 and/or to the remote computer device 145 continuously, at scheduled intervals, when requested, and/or when certain conditions are satisfied (e.g., during an alarm condition).

The remote computer device 145 may be a computing entity operable to enable a remote user to monitor the output of the sensor units 110. The remote computer device 145 may be functionally and/or structurally similar to the local computer devices 115, 120 and may be operable to receive data streams from and/or send signals to at least one of the sensor units 110 via the network 125. The network 125 may be the Internet, an intranet, a personal area network, a local area network (LAN), a wide area network (WAN), a virtual network, a telecommunications network implemented as a wired network and/or wireless network, etc. The remote computer device 145 may receive and/or send signals over the network 125 via communication links 150 and server 135.

The remote computer device 145 may be used by, for example, a health care professional to monitor the output of the sensor units 110. In some embodiments, as described in further detail herein, the remote computer device 145 may receive an indication of physiological data when the sensors detect an alert condition, when the healthcare provider requests the information, at scheduled intervals, and/or at the request of the healthcare provider and/or the person 105. For example, the remote computer device 145 may be operable to receive summarized physiological data from the server 135 and display the summarized physiological data in a convenient format. The remote computer device 145 may be located, for example, at a nurses station or in a patient's room, and configured to display a summary of the physiological data collected from one or more patients. In some instances, the local computer devices 115, 120 may also be operable to receive and display physiological data in much the same way that the remote computer device 145 is operable.

The server 135 may be configured to communicate with the sensor units 110, the local computer devices 115, 120, third-party sensors 130, the remote computer device 145 and databases 140. The server 135 may perform additional processing on signals received from the sensor units 110, local computer devices 115, 120 or third-party sensors 130, or may simply forward the received information to the remote computer device 145 and databases 140. The databases 140 may be examples of electronic health records (“EHRs”) and/or personal health records (“PHRs”), and may be provided by various service providers. The third-party sensor 130 may be a sensor that is not attached to the person 105 but that still provides data that may be useful in connection with the data provided by sensor units 110. In certain embodiments, the server 135 may be combined with one or more of the local computer devices 115, 120 and/or the remote computer device 145.

The server 135 may be a computing device operable to receive data streams (e.g., from the sensor units 110 and/or the local computer devices 115, 120), store and/or process data, and/or transmit data and/or data summaries (e.g., to the remote computer device 145). For example, the server 135 may receive a stream of heart rate data from a sensor unit 110, a stream of oxygen saturation data from the same or a different sensor unit 110, and a stream of body temperature data from either the same or yet another sensor unit 110. In some embodiments, the server 135 may “pull” the data streams, e.g., by querying the sensor units 110 and/or the local computer devices 115, 120. In some embodiments, the data streams may be “pushed” from the sensor units 110 and/or the local computer devices 115, 120 to the server 135. For example, the sensor units 110 and/or the local computer devices 115, 120 may be configured to transmit data as it is generated by or entered into that device. In some instances, the sensor units 110 and/or the local computer devices 115, 120 may periodically transmit data (e.g., as a block of data or as one or more data points).

The server 135 may include a database (e.g., in memory) containing physiological data received from the sensor units 110 and/or the local computer devices 115, 120. Additionally, as described in further detail herein, software (e.g., stored in memory) may be executed on a processor of the server 135. Such software (executed on the processor) may be operable to cause the server 135 to monitor, process, summarize, present, and/or send a signal associated with physiological data.

Although the server 135 and the remote computer device 145 are shown and described as separate computing devices, in some embodiments, the remote computer device 145 performs the functions of the server 135 such that a separate server 135 may not be necessary. In such an embodiment, the remote computer device 145 receives physiological data streams from the sensor units 110 and/or the local computer devices 115, 120, processes the received data, and displays the processed data as summarized physiological data.

Additionally, although the remote computer device 145 and the local computer devices 115, 120 are shown and described as separate computing devices, in some embodiments, the remote computer device 145 performs the functions of the local computer devices 115, 120 such that a separate local computer device 115, 120 may not be necessary. In such an embodiment, the user (e.g., a nurse or a doctor) may manually enter the patient's physiological data (e.g., the patient's body temperature) directly into the remote computer device 145.

In the system 100 of FIG. 1, a sensor unit 110 may, for example, generate a data point associated with a patient's respiratory rate every hour on the hour as well as every twenty minutes past the hour and every twenty minutes before the hour. The same or a different sensor unit 110 may, for example, generate a data point associated with the patient's blood oxygen saturation every half-hour, at the half-hour, ten minutes before the hour, and ten minutes past the hour, and a nurse may enter the patient's body temperature data via the local computer device 115, 120 irregularly but, for example, approximately six times daily. If these collected data points were time stamped and presented in a table without any processing, then no one row in the table would include data points for all measured parameters. For example, the row time stamped at “4:30 pm” would only have a data point for the patient's blood oxygen saturation level; the cells in this row associated with the patient's respiratory rate and body temperature would be empty or have null values because no data points for these particular parameters were collected at the time stamped time. The local computer devices 115, 120, the server 135 and/or the remote computer device 145 may process the data points collected by the sensor units 110 and/or the local computer devices 115, 120 to produce a summary of the data such that the local computer devices 115, 120 and/or the remote computer device 145 may display a summary row that includes a data point for each parameter. In other words, no cell in the summary row is empty or has a null value if data associated with the physiological parameter represented by that cell's column has been received.

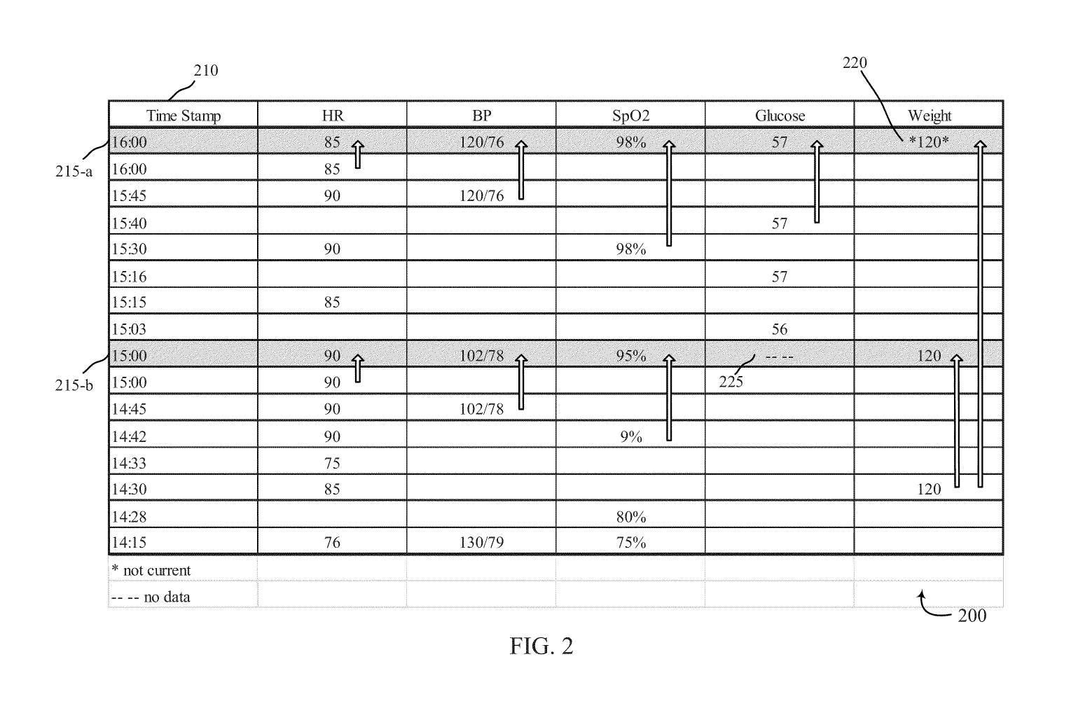

The summary row may correspond to an epoch, or a designated period of time. An epoch may be chosen such that the data to be displayed in the epoch is physiologically meaningful, meaning that the length of a selected epoch may be chosen so as to not be too short or too long, based on the physiological parameters to be displayed. Example epochs are discussed in relation to FIG. 2.

FIG. 2 is a graphical representation of a table 200 of unsynchronized physiological data that has been summarized by epoch, in accordance with various embodiments. In table 200, data values associated with five separate data streams are illustrated. The data streams may have been collected via sensor units 110 and/or local computer devices 115, 120 of system 100 of FIG. 1. In table 200, the data streams represent five different physiological parameters. The physiological parameters illustrated in FIG. 2 include a patient's heart rate (HR), blood pressure (BP), oxygen saturation (SpO2), glucose and weight. The data streams for the five physiological parameters are collected asynchronously, as is demonstrated by the inconsistency of entries in table 200. For example, each data point includes a time stamp 210. As illustrated, the heart rate data stream includes a data point every fifteen minutes, while the blood pressure data stream includes a data point every thirty minutes. The heart rate and blood pressure data streams may be generated, for example, by automatic monitoring devices, such as the sensor units 110 shown and described above with reference to FIG. L The data points and corresponding time stamps on the table 200 for the patient's oxygen saturation and glucose levels indicate that these data streams are received at irregular intervals, while the weight data consists of a single data point. The patient's oxygen saturation, glucose, and/or weight data could be manually entered data or could be received on an on-demand basis from sensor units 110. For example, an observer, such as a nurse may take a measurement and enter the data via one of the local computer devices 115, 120, or an observer at a remote computer device 145 could send a request to a sensor unit 110, asking the sensor unit to provide at the time of the request specific physiological data.

As illustrated in table 200, the patient's physiological data is summarized using one-hour epochs, represented by shaded epoch rows 215 which include summary data that may provide an overview of the patient's vital signs during the epoch (i.e., in this case, the one hour time period). The summary data displayed in the shaded epoch rows 215 includes the most physiologically relevant data point for each of the patient's vital signs. In particular, epoch row 215-a includes the most physiologically relevant values for each of the parameters represented by the five different data streams during the epoch spanning from 15:00 to 16:00. Epoch row 215-b includes the most physiologically relevant values for each of the parameters represented by the five different data streams during the epoch spanning from 14:00 to 15:00.

For each of the different physiological parameters represented in table 200, one or more different methods may be used to determine the most physiologically relevant value. For example, one method could include using the most recent value. This method is used for each of the physiological parameters represented in table 200. Another method may include determining an average or a median of values within the epoch. An additional method may include determining an average or a median of most recent values within the epoch. In each method, values may also be evaluated to determine if the values are useful. For example, if a physician is interested in an at-rest heart rate, then only heart rate values that represent an at-rest heart rate are to be considered in the selection or determination of a single summary heart rate value for the epoch.

At times, the collected data may be insufficient to allow a determination of a summary data value for each physiological parameter using only data values from a particular epoch. For example, in table 200, no weight values were collected during the epoch extending from 15:00 to 16:00. In this case, the most physiologically relevant weight value from a different epoch may be used to populate the epoch row 215-a. An indication 220 (shown as “* *”) is used to show that the data point is not current (or not determined from the corresponding epoch). As another example, there may be times when there are no physiologically relevant data values available at all, whether inside or outside of a given epoch. An example of this is shown in epoch row 215-b, where the summary value for the patient's glucose levels is left blank because there is no available data from which to derive a summary value. In this case, an indication 225 (shown as “-- --”) is used to indicate that no data is available for glucose.

The summary data from each epoch row 215 may be determined and output at any one of the local computer devices 115, 120 or the remote computer device 145 of system 100. The determining and outputting summary data, a clinician is enabled to see and understand an overview of multiple asynchronous data streams on a single line. A graphical user interface could even be provided at either the local computer devices 115, 120 or the remote computer device 145 to present a summary of unsynchronized data streams for multiple patients, wherein summarized data for each patient is provided on a single line. In this way, an observer, such as a nurse, may view a compact summary display that provides an overview of multiple asynchronous data streams for multiple patients.

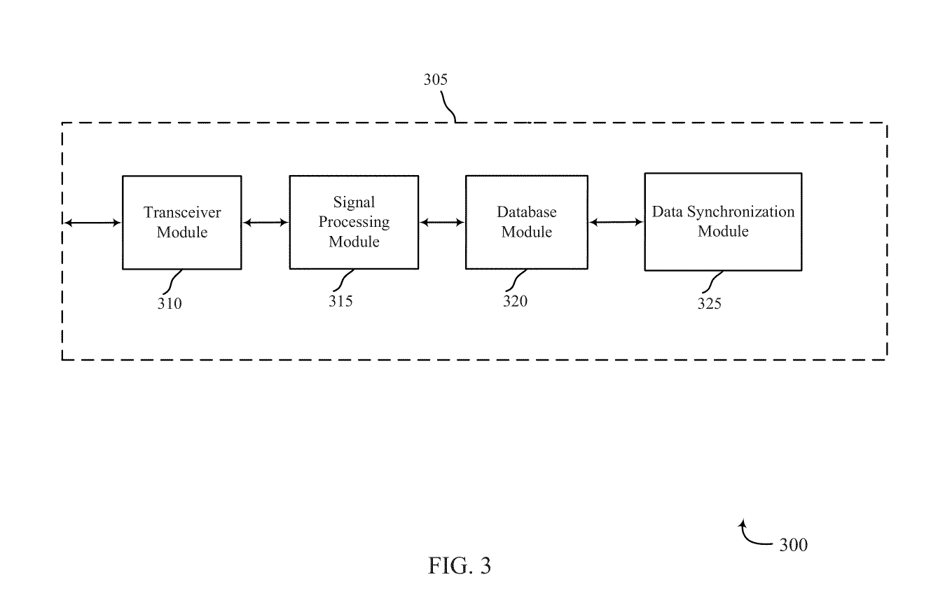

FIG. 3 shows a block diagram 300 that includes apparatus 305, which may be an example of one or more aspects of the local computer devices 115, 120 and/or remote computer device 145 (of FIG. 1) for use in physiological monitoring, in accordance with various aspects of the present disclosure. In some examples, the apparatus 305 may include a transceiver module 310, a signal processing module 315, a database module 320, and a data synchronization module 325. Each of these components may be in communication with each other.

The components of the apparatus 305 may, individually or collectively, be implemented using one or more application-specific integrated circuits (ASICs) adapted to perform some or all of the applicable functions in hardware. Alternatively, the functions may be performed by one or more other processing units (or cores), on one or more integrated circuits. In other examples, other types of integrated circuits may be used (e.g., Structured/Platform ASICs, Field Programmable Gate Arrays (FPGAs), and other Semi-Custom ICs), which may be programmed in any manner known in the art. The functions of each unit may also be implemented, in whole or in part, with instructions embodied in a memory, formatted to be executed by one or more general or application-specific processors.

In some examples, the transceiver module 310 may be operable to receive data streams from the sensor units 110, as well as to send and/or receive other signals between the sensor units 110 and either the local computer devices 115, 120 or the remote computer device 145 via the network 125 and server 135. In an embodiment, the transceiver module 310 may receive data streams from the sensor units 110 and also forward the data streams to other devices. The transceiver module 310 may include wired and/or wireless connectors. For example, in some embodiments, sensor units 110 may be portions of a wired or wireless sensor network, and may communicate with the local computer devices 115, 120 and/or remote computer device 145 using either a wired or wireless network. The transceiver module 310 may be a wireless network interface controller (“NIC”), Bluetooth® controller, IR communication controller, ZigBee® controller and/or the like.

In some examples, the signal processing module 315 includes circuitry, logic, hardware and/or software for processing the data streams received from the sensing units 110. The signal processing module 315 may include filters, analog-to-digital converters and other digital signal processing units. Data processed by the signal processing module 315 may be stored in a buffer, for example, in the database module 320. The database module 320 may include magnetic, optical or solid-state memory options for storing data processed by the signal processing module 315.

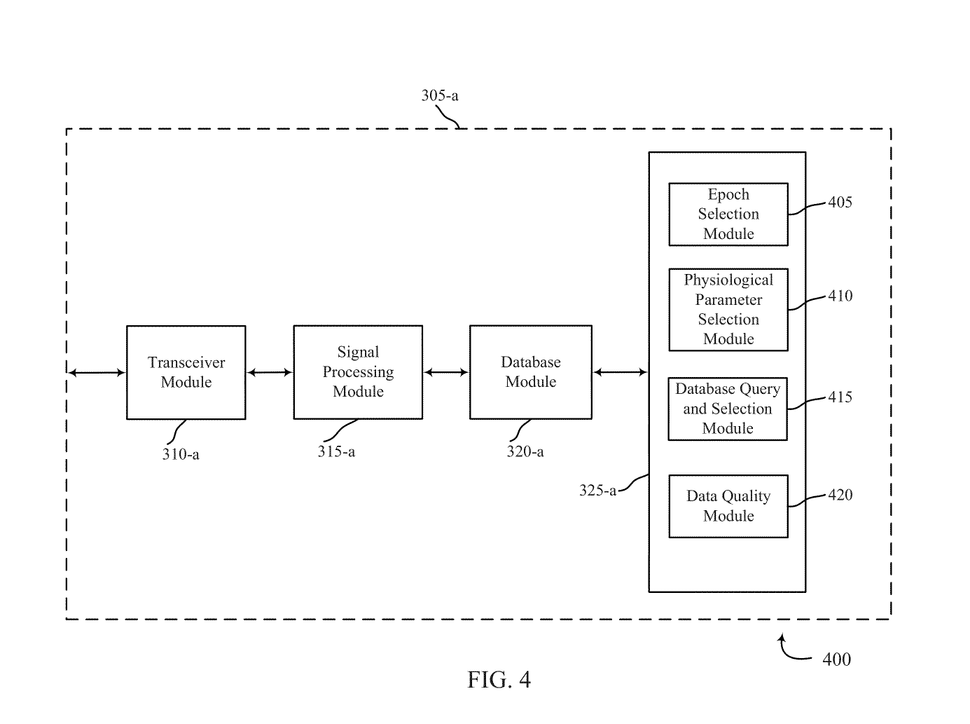

The data synchronization module 325 may access the data stored in the database module 320 and output the stored data in a meaningful summary, as presented, for example, in the table 200 of FIG. 2. The data synchronization module 325, then, takes unsynchronized data streams and, for each selected epoch, determines physiologically meaningful summary values for each parameter represented by the data streams. The process is explained in greater detail with respect to FIGS. 4 and 8, detailed below.

FIG. 4 shows a block diagram 400 that includes apparatus 305-a, which may be an example of apparatus 305 (of FIG. 3), in accordance with various aspects of the present disclosure. In some examples, the apparatus 305-a may include a transceiver module 310-a, a signal processing module 315-a, a database module 320-a, and a data synchronization module 325-a, which may be examples of the transceiver module 310, the signal processing module 315, the database module 320 and the data synchronization module 325 of FIG. 3. In some examples, the data synchronization module 325-a may include an epoch selection module 405, a physiological parameter selection module 410, a database query and selection module 415, and a data quality module 420. The modules 405, 410, 415 and/or 420 may each be used in aspects of synchronizing summary data from unsynchronized data streams. Additionally, while FIG. 4 illustrates a specific example, the functions performed by each of the modules 405, 410, 415 and/or 420 may be combined or implemented in one or more other modules.

The epoch selection module 405 may be used to select or determine an appropriate epoch. In situations where received data streams are asynchronous, data values from the received data streams may be summarized within epochs, or periods of time. In this way, data for multiple physiological parameters may be on a row of a table of data and associated with a specific epoch even if some or all of the physiological measurements were taken at different moments in time, thus each having a different time stamp. The length of the epoch may be chosen such that physiological data received at the beginning of the epoch remains medically relevant at the end of the epoch. For example, in some instances, a data point associated with blood glucose levels may only be medically relevant for four hours. Accordingly, the epoch may be selected to be less than four hours such that an epoch does not include a glucose measurement older than four hours.

In addition or alternatively, the epoch may be selected such that at least one data point from each data stream is likely to be received during the epoch. In other words, the length of an epoch may be selected to be equal to or longer than the sampling frequency of the physiological parameter sampled least frequently. For example, if data associated with the patient's body temperature is manually entered once an hour and the other monitored parameters are automatically collected by sensors once every second, then the epoch may be selected to be 80 minutes, for example, so that at least one data point corresponding to the patient's body temperature is included in the epoch. As such, the number of epochs for which there is no temperature data may be minimized.

In some embodiments, a caregiver may choose the length of the epoch by selecting or entering an epoch length into either the local computer device 115, 120 or the remote computer device 145. In other embodiments, the length of the epoch may be preselected. For example, software executing on the processor of the local computer device 115, 120, the remote computer device 145, or the server 135 may pre-select the epoch length. In some embodiments selecting the length of the epoch may include balancing competing concerns of relevancy and sampling rate.

The physiological parameter selection module 410 may be used to determine which data stream to analyze in order to a summary value for the physiological parameter corresponding to the data stream. The physiological parameter selection module 410 may increment through each of the received data streams such that a summary value is selected for each corresponding physiological parameter. Alternatively, the physiological parameter selection module 410 may be used to selectively evaluate only certain of the received data streams—for example, those data streams corresponding to the physiological parameters of most interest to a clinician.

The database query and selection module 415 may be used to obtain for evaluation at least a portion of the data stream corresponding to a selected physiological parameter. Once an epoch and a physiological parameter are selected, relevant data may be fetched from a database or other storage device. For example, software executing on the processor of either the local computer device 115, 120, the remote computer device 145 or the server 135 may search the database or other storage device for data matching the selected epoch and the selected physiological parameter.

The database query and selection module 415 may also be used to select or determine a summary value for each of the selected physiological parameters and epochs. The summary value may be selected or determined from the values obtained from the database or other storage device. Summary values may be selected or determined based on criteria that may provide the most physiologically relevant summary value for each parameter or data stream. For example, in some cases, the most physiologically relevant value may be the most recently recorded value. In other cases, the most physiologically relevant value may be an average or a median of the selected data. In still other cases, the most physiologically relevant vale may be an average or a median of the most recent values for a data stream.

The selected or determined summary values may also be evaluated based on their quality. For example, values in data streams that represent vital signs during at-rest conditions may have a higher quality and relevance than values that represent vital signs during active conditions, depending on the needs of the monitoring clinician.

The data quality module 420 may be used to evaluate the quality of the data points or values determined or selected as summary values. Evaluating the quality of the data point may include examining metadata associated with the data point. In some embodiments, a monitoring device may be operable to evaluate the quality of a measurement and send an indication of measurement confidence. The indication of measurement confidence may be stored in the database. The measurement confidence may be compared with a confidence threshold. For example, a sensor unit 110 may include a pulse oximeter which, in addition to measuring oxygen saturation, may also measure and report metadata such as signal strength. The signal strength may be used as an indication of the confidence of the measurement. The reported signal strength may also be compared with a threshold signal strength to determine if the signal quality is sufficient. In addition or alternatively, selected or determined summary values may be compared with recent or historical ranges of the summary value. For example, software executing on the processor of the local computer device 115, 120, remote computer device 145 or server 135 may assign a low quality indicator to a selected measurement of patient weight if the selected measurement differs by more than 10% from a measurement taken within the last 24 hours. Similarly, a selected measurement of respiratory rate may be assigned a low quality if it exceeds 200 breaths per minute and if the patient is of sufficient age that 200 breaths per minute is not physically possible.

If the quality of the selected data point exceeds the quality threshold, an indication of the selected or determined summary value may be output or displayed or sent to another device for display. For instance, one of the local computer devices 115, 120, the remote computer device 145 or the server 135 may send or output an indication of the data point, an indication of the epoch, and/or the time stamp. The displaying device may then display the data point in a row of the table labeled with the epoch in a column labeled with the physiological parameter.

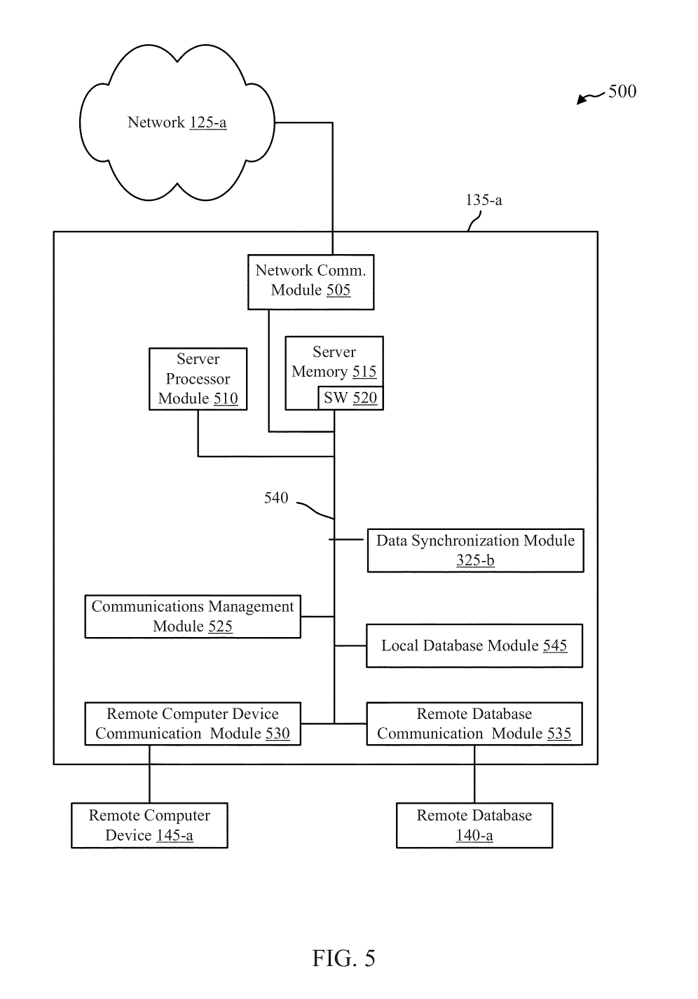

FIG. 5 shows a block diagram 500 of a server 135-a for use in summarizing asynchronous data streams, in accordance with various aspects of the present disclosure. In some examples, the server 135-a may be an example of aspects of the server 135 described with reference to FIG. 1. In other examples, the server 135-a may be implemented in either the local computer devices 115, 120 or the remote computer device 145 of FIG. 1. The server 135-a may be configured to implement or facilitate at least some of the features and functions described with reference to the server 135, the local computer devices 115, 120 and/or the remote computer device 145 of FIG. 1.

The server 135-a may include a server processor module 510, a server memory module 515, a local database module 545, and/or a communications management module 525. The server 135-a may also include one or more of a network communication module 505, a remote computer device communication module 530, and/or a remote database communication module 535. Each of these components may be in communication with each other, directly or indirectly, over one or more buses 540.

The server memory module 515 may include RAM and/or ROM. The server memory module 515 may store computer-readable, computer-executable code 520 containing instructions that are configured to, when executed, cause the server processor module 510 to perform various functions described herein related to presenting asynchronous data stream values. Alternatively, the code 520 may not be directly executable by the server processor module 510 but be configured to cause the server 135-a (e.g., when compiled and executed) to perform various of the functions described herein.

The server processor module 510 may include an intelligent hardware device, e.g., a central processing unit (CPU), a microcontroller, an ASIC, etc. The server processor module 510 may process information received through the one or more communication modules 505, 530, 535. The server processor module 510 may also process information to be sent to the one or more communication modules 505, 530, 535 for transmission. Communications received at or transmitted from the network communication module 505 may be received from or transmitted to sensor units 110, local computer devices 115, 120, or third-party sensors 130 via network 125-a, which may be an example of the network 125 described in relation to FIG. 1. Communications received at or transmitted from the remote computer device communication module 530 may be received from or transmitted to remote computer device 145-a, which may be an example of the remote computer device 145 described in relation to FIG. 1. Communications received at or transmitted from the remote database communication module 535 may be received from or transmitted to remote database 140-a, which may be an example of the remote database 125 described in relation to FIG. 1. Additionally, a local database may be accessed and stored at the server 135-a. The local database module 545 is used to access and manage the local database, which may include data received from the sensor units 110, the local computer devices 115, 120, the remote computer devices 145 or the third-party sensors 130 (of FIG. 1).

The server 135-a may also include a data synchronization module 325-b, which may be an example of the data synchronization module 325 of apparatus 305 described in relation to FIGS. 3 and/or 4. The data synchronization module 325-b may perform some or all of the features and functions described in relation to the data synchronization module 325, including selecting an epoch, selecting physiological parameters to summarize, selecting and obtaining from either the local database module 545 or the remote database 140-a data corresponding to the selected epoch and physiological parameter, determining a summary value for the selected epoch and physiological parameter, and ensuring the quality of the determined summary value.

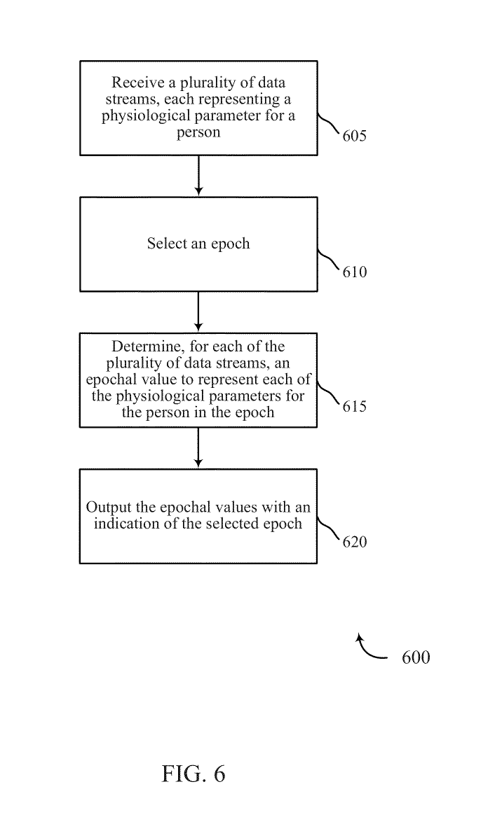

FIG. 6 is a flow chart illustrating an example of a method 600 for outputting unsynchronized data streams, in accordance with various aspects of the present disclosure. For clarity, the method 600 is described below with reference to aspects of one or more of the local computer devices 115, 120, remote computer device 145, and/or server 135 described with reference to FIGS. 1, and/or 5, or aspects of one or more of the apparatus 305 described with reference to FIGS. 3 and/or 4. In some examples, a local computer device, remote computer device or server such as one of the local computer devices 115, 120, remote computer device 145, server 135 and/or an apparatus such as one of the apparatuses 305 may execute one or more sets of codes to control the functional elements of the local computer device, remote computer device, server or apparatus to perform the functions described below.

At block 605, the method 600 may include receiving a plurality of data streams, each representing a physiological parameter for a person. the plurality of data streams may be received from one or more sensor units, for example.

At block 610, the method 600 may include selecting an epoch. As described above, an epoch may be selected based on the frequency that different physiological parameters are measured, for example, as well as based on the time-based relevancy of the physiological parameters being measured.

At block 615, the method 600 may include determining, for each of the plurality of data streams, an epochal value to represent each of the physiological parameters for the person in the epoch. The epochal value is a summary value that is physiologically relevant based on the physiological parameter represented by the summary value. In other words, the epochal or summary value is determined using a method that is appropriate for the physiological parameter being measured. Methods may include selecting the most recently measured data point, determining an average or a median of a collection of data points, and/or determining an average or a median of the most recent data points in a collection of data points.

At block 620, the method 600 may include outputting the epochal values with an indication of the selected epoch. This may be performed using a tabular format, as is illustrated in table 200 of FIG. 2. The epochal or summary values are presented such that, as long as quality data exists for each physiological parameter, the epoch rows include physiologically relevant summary values for each physiological parameter represented by the received data streams.

In some embodiments, the operations at blocks 605, 610, 615 or 620 may be performed using the data synchronization module 325 described with reference to FIGS. 3, 4 and/or 5. Nevertheless, it should be noted that the method 600 is just one implementation and that the operations of the method 600 may be rearranged or otherwise modified such that other implementations are possible.

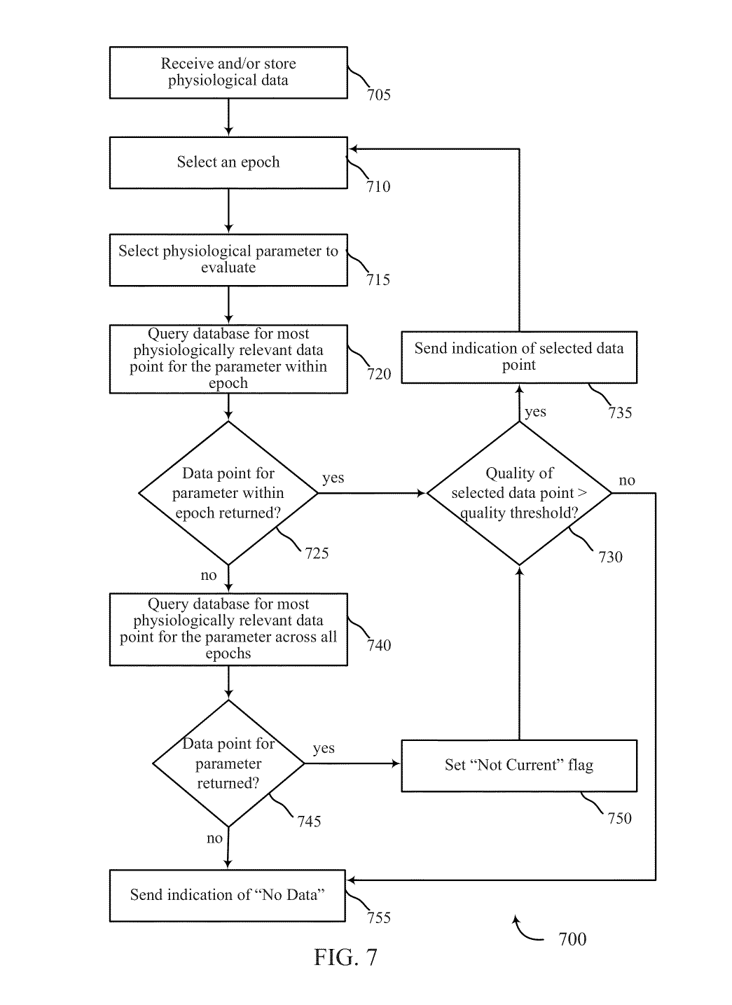

FIG. 7 is a flow chart illustrating an example of a method 700 for outputting unsynchronized data streams, in accordance with various aspects of the present disclosure. For clarity, the method 700 is described below with reference to aspects of one or more of the local computer devices 115, 120, remote computer device 145, and/or server 135 described with reference to FIGS. 1, and/or 5, or aspects of one or more of the apparatus 305 described with reference to FIGS. 3 and/or 4. In some examples, a local computer device, remote computer device or server such as one of the local computer devices 115, 120, remote computer device 145, server 135 and/or an apparatus such as one of the apparatuses 305 may execute one or more sets of codes to control the functional elements of the local computer device, remote computer device, server or apparatus to perform the functions described below.

The method 700 may be used, for example, to monitor one or more of a patient's vital signs (or other physiological statistics) and to present the associated data to a healthcare professional (e.g., a nurse or doctor) in a summarized tabular format that is easy to read. In some embodiments, the patient may be monitored in a hospital, a hospice or other healthcare related facility. In other embodiments, the patient may be monitored at home and the patient's physiological data may be streamed to the location of the healthcare provider. The vital signs or other physiological parameters being monitored may include, but are not limited to, the patient's heart rate, respiratory rate, activity level (e.g., standing, sitting, laying, walking, etc.), body temperature, blood pressure, blood oxygen saturation, weight, blood sugar, and/or the like.

As shown in FIG. 7, at step 705, the method 700 includes receiving and/or storing physiological data. For example, the local computer devices 115, 120, remote computer device 145 and/or server 135 shown and described above with reference to FIG. 1 may receive one or more data streams from the sensor units 110 and/or the local computer devices 115, 120 and may store the received data, for example, in a database. Each of the received data streams may be associated with a different physiological parameter and may be received from one or more sensor units. For example, the server may receive a stream of patient's heart rate data from an ECG sensor worn by the patient, and a stream of the patient's weight data that was manually entered at the local computer devices.

In some embodiments, each of the streamed data points is time stamped. Similarly stated, a time code may be associated with each streamed data point. The time code may correspond, for example, to the time the data was collected by a sensor unit, the time the data was manually entered into a local computer device or remote computer device, and/or the time when the data was received by the server.

In some embodiments, the received data streams are asynchronous. For example, in some embodiments, data associated with the patient's heart rate is received more frequently than data associated with the patient's weight. In embodiments where the patient's physiological data is received at either a local computer device, a remote computer device, or a server, the local computer device, remote computer device and/or server may be operable to summarize the received physiological data over an epoch such that an overview of multiple physiological parameters may be presented to a healthcare provider. In this way, data for multiple physiological parameters may be presented on a row associated with a specific time stamp (an epoch time stamp) even though some, but not all, of the physiological measurements were taken at the time identified by the epoch time stamp.

The asynchronous data streams may be summarized over an epoch (i.e., a period of time). The length of the epoch may be selected (at step 710) such that physiological data received at the beginning of the epoch remains medically relevant at the end of the epoch. For example, in some instances, a data point associated with blood glucose may only be medically relevant for four hours. Accordingly, the epoch may be selected to be less than four hours such that an epoch does not include a glucose measurement older than four hours.

In addition or alternatively, the epoch may be selected such that at least one data point from each data stream is likely to be received during the epoch. Similarly stated, the length of an epoch may be selected to be equal to or longer than the sampling frequency of the physiological parameter sampled least frequently. For example, if data associated with the patient's body temperature is manually entered once an hour and the other monitored parameters are automatically collected by sensors once every second, then the epoch may be selected to be 80 minutes so that at least one data point corresponding to the patient's body temperature is included in the epoch. As such, the number of epochs for which there is no temperature data may be minimized.

In some embodiments, a caregiver chooses the length of the epoch. For example, the caregiver, using the local computer devices 115, 120 or remote computer device 145, may determine and enter the length of time for which physiological data is relevant. In other embodiments, the length of the epoch may be preselected. For example, software executing on the processor of the local computer devices 115, 120, the remote computer device 145, and/or the server 135 may pre-select the epoch length.

In some embodiments selecting the length of the epoch may include balancing competing concerns of relevancy and sampling rate. For instance, in an embodiment where there is a small possibility that a respiratory rate data point will no longer be medically relevant 10 minutes after being recorded and temperature is measured only once an hour, the epoch time may be set to 15 minutes, for example.

Step 710 of method 700 may be performed by a processor of either the local computer devices 115, 120, the remote computer device 145 and/or the server 135. The method 700 may also include a step of selecting a physiological parameter to be evaluated, at step 715. Selecting the epoch, at step 710, and selecting the physiological parameter, at step 715, may be analogous to selecting a row and a column, respectively, of a table containing a summary of asynchronous data. As described in further detail herein, the method may be iterated such that each epoch and parameter are evaluated sequentially (although, in other embodiments, epochs and/or parameters may be evaluated in parallel).

Once the physiological parameter has been selected, at step 715, a database having data associated with the collected physiological data may be queried for the most physiologically relevant data point associated with the parameter within the epoch, at step 720. For example, software executing on the processor of the local computer devices 115, 120, the remote computer device 145, and/or the server 135 may search the database for data matching the epoch selected at step 710 and the parameter selected at step 715, and may select or determine the data point that is most physiologically relevant. Physiologically relevant data points may include the data point having the most recent time stamp, an average or median data point of the data corresponding to the selected epoch and physiological parameter, or an average or median data point of the most recent data corresponding to the selected epoch and physiological parameter.

The method 700 may include determining whether a data point associated with the epoch selected at step 710 and the parameter selected at step 715 was returned, at step 725. If such a data point was returned, the quality of the data point may be evaluated, at step 730 (e.g., code executing on the processor of the local computer device, remote computer device or server may evaluate the quality of the data point). Evaluating the quality of the data point may include examining metadata associated with the data point. In some embodiments, a monitoring device may be operable to evaluate the quality of a measurement and send an indication of measurement confidence. The indication of measurement confidence may be stored in the database. The method may include comparing this measurement confidence to a confidence threshold, at step 730. For example, a pulse oximeter, in addition to measuring oxygen saturation, may measure and report, as metadata, signal strength, which may be an indication of the confidence of the measurement. In such an example, the method may include comparing the signal strength to a threshold signal strength, at step 730. In addition or alternatively, the method may include comparing the data point to recent or historical ranges of the parameter. For example, software executing on the processor of the local computer devices, remote computer device and/or server may assign a low quality indicator to a selected measurement of patient weight if the selected measurement differs by more than 10% from a measurement taken within the last 24 hours. Similarly, a selected measurement of respiratory rate may be assigned a low quality if it exceeds 200 breaths per minute and if the patient is unlikely to be able to breath that frequently.

If the quality of the selected data point exceeds the quality threshold, at step 730, an indication of the selected data point may be sent, at step 735. The indication of the selected data point may be sent from a local computer device or server to a remote computer device. Alternatively, the indication of the selected data point may be output by a local computer device or remote computer device. In this way, either the local computer devices or remote computer device may be operable to display the selected data point as associated with the epoch. For instance, the local computer devices or server may send an indication of the data point, an indication of the epoch, and/or the time stamp. The local computer devices or remote computer device may then display the data point in a row of the table labeled with the epoch in a column labeled with the physiological parameter.

If no data point matching the epoch selected at step 710 and the parameter selected at step 715 is returned at step 725, then the database may be queried (e.g., software executing on the processor of either the local computer devices, the remote computer device or the server may query the database) for any data point associated with the parameter selected, at step 740. Software executing on the processor of the local computer devices, remote computer device or server may include determining whether any data for the parameter selected at step 715 is stored within the database, at step 745. If the query at step 740 returns a data point, the processor may set a “not current” flag, at step 750, to indicate that the selected data point is not associated with the epoch selected at step 710. The selected data point and an indication of the “not current flag” may then be sent, for example, from the local computer devices or server and/or output by either the local computer devices or remote computer device, at step 735. In this way, the local computer devices or remote computer device may display a data point associated with the parameter and may indicate that the data is not of the current epoch by, for example, using an asterisk, caption, presenting the data in an alternate color, etc.

If, however, it is determined at step 745 that the database contains no data associated with the parameter selected at step 715, or if at step 730 the quality of the selected data point does not exceed the quality threshold, a “no data” signal may be sent, at step 755, for example, from the local computer devices or server and/or output by the local computer devices or remote computer device. In this way, the local computer devices and/or remote computer device may provide an indication to the user that no data associated with the parameter is available.

Having either sent the indication of “no data,” at step 755, or sent the indication of a selected data point, at step 735, the method 700 may return to select an epoch, at step 710. Selecting the epoch, at step 710, may be analogous to selecting a row of a table of summary data. If the row is complete (e.g., the processor has iterated for each parameter within the row), a new epoch may be selected, at step 710. Selecting the new epoch, at step 710, may include selecting a new time period and/or a new patient to evaluate. For example, having summarized all the parameters for the patient for the epoch, the processor may include iterating to evaluate the physiological data of another patient.

In some instances, selecting the epoch, at step 710, may include selecting the same epoch. For example, if the row represented by the epoch is not complete (e.g., the method 700 has not iterated through for each parameter), the processor may select the same epoch and a new parameter may be selected, at step 715. Similarly stated, the column of the summary table 200 (of FIG. 2) may be iterated.

The above description provides examples, and is not limiting of the scope, applicability, or configuration set forth in the claims. Changes may be made in the function and arrangement of elements discussed without departing from the spirit and scope of the disclosure. Various embodiments may omit, substitute, or add various procedures or components as appropriate. For instance, the methods described may be performed in an order different from that described, and various steps may be added, omitted, or combined. Also, features described with respect to certain embodiments may be combined in other embodiments.

The detailed description set forth above in connection with the appended drawings describes exemplary embodiments and does not represent the only embodiments that may be implemented or that are within the scope of the claims. The term “exemplary” used throughout this description means “serving as an example, instance, or illustration,” and not “preferred” or “advantageous over other embodiments.” The detailed description includes specific details for the purpose of providing an understanding of the described techniques. These techniques, however, may be practiced without these specific details. In some instances, well-known structures and devices are shown in block diagram form in order to avoid obscuring the concepts of the described embodiments.

Information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, multiple microprocessors, one or more microprocessors in conjunction with a DSP core, or any other such configuration. A processor may in some cases be in electronic communication with a memory, where the memory stores instructions that are executable by the processor.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope and spirit of the disclosure and appended claims. For example, due to the nature of software, functions described above may be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items indicates a disjunctive list such that, for example, a list of “at least one of A, B, or C” means A or B or C or AB or AC or BC or ABC (i.e., A and B and C).

A computer program product or computer-readable medium both include a computer-readable storage medium and communication medium, including any mediums that facilitates transfer of a computer program from one place to another. A storage medium may be any medium that may be accessed by a general purpose or special purpose computer. By way of example, and not limitation, computer-readable medium may comprise RAM, ROM, EEPROM, CD-ROM or other optical disk storage, magnetic disk storage or other magnetic storage devices, or any other medium that may be used to carry or store desired computer-readable program code in the form of instructions or data structures and that may be accessed by a general-purpose or special-purpose computer, or a general-purpose or special-purpose processor. Also, any connection is properly termed a computer-readable medium. For example, if the software is transmitted from a website, server, or other remote light source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include compact disc (CD), laser disc, optical disc, digital versatile disc (DVD), floppy disk and blu-ray disc where disks usually reproduce data magnetically, while discs reproduce data optically with lasers. Combinations of the above are also included within the scope of computer-readable media.

The previous description of the disclosure is provided to enable a person skilled in the art to make or use the disclosure. Various modifications to the disclosure will be readily apparent to those skilled in the art, and the generic principles defined herein may be applied to other variations without departing from the spirit or scope of the disclosure. Throughout this disclosure the term “example” or “exemplary” indicates an example or instance and does not imply or require any preference for the noted example. Thus, the disclosure is not to be limited to the examples and designs described herein but is to be accorded the widest scope consistent with the principles and novel features disclosed herein.

1. A method for outputting unsynchronized physiological data, the method comprising:
receiving a plurality of data streams, each representing a physiological parameter for a person;
selecting an epoch;
determining, for each of the plurality of data streams, an epochal value to represent each of the physiological parameters for the person in the epoch; and
outputting the epochal values with an indication of the selected epoch.

2. The method of claim 1, wherein determining the epochal values representing each of the physiological parameters comprises:
selecting, for at least one of the physiological parameters, the corresponding epochal value from a plurality of values included in the corresponding plurality of data streams.

3. The method of claim 2, wherein selecting the corresponding epochal value comprises:
identifying values from the plurality of values included in the corresponding plurality of data streams that are within the epoch; and
using only the values that are within the epoch to determine the epochal value.

4. The method of claim 3, further comprising:
using only the most recent values that are within the epoch to determine the epochal value.

5. The method of claim 3, further comprising:
mathematically computing the epochal value from the values that are within the epoch.

6. The method of claim 2, wherein selecting the corresponding epochal value comprises:
identifying values from the plurality of values included in the corresponding plurality of data streams that are not within the epoch; and
using the values that are not within the epoch to determine the epochal value.

7. The method of claim 6, wherein outputting the epochal values comprises:
outputting an indication that the epochal values selected using values that are not within the epoch are not current.

8. The method of claim 1, wherein outputting the epochal values comprises:
outputting one epochal value for each of the physiological parameters represented by the plurality of data streams.

9. The method of claim 1, wherein determining the epochal values representing each of the physiological parameters comprises:
presenting an option to a user to select, for at least one of the physiological parameters, a physiologically relevant algorithm for determining the corresponding epochal value from among a plurality of algorithms.

10. The method of claim 1, wherein selecting the epoch comprises:
determining, for each of the physiological parameters, a physiologically relevant period of time; and
selecting a shortest of the determined physiologically relevant periods of time as the epoch.

11. The method of claim 1, wherein outputting the epochal values comprises:
displaying the epochal values in table form.

12. An apparatus for outputting unsynchronized physiological data, comprising:
a processor;
memory in electronic communication with the processor; and
instructions stored in the memory, the instructions being executable by the processor to:
receive a plurality of data streams each representing a physiological parameter for a person;
select an epoch;
determine, for each of the plurality of data streams, an epochal value to represent each of the physiological parameters for the person in the epoch; and
output the epochal values with an indication of the selected epoch.

13. The apparatus of claim 12, wherein the instructions for determining the epochal values representing each of the physiological parameters are executable by the processor to:
select, for at least one of the physiological parameters, the corresponding epochal value from a plurality of values included in the corresponding plurality of data streams.

14. The apparatus of claim 13, wherein the instructions for selecting corresponding epochal values are executable by the processor to:
identify values from the plurality of values included in the corresponding plurality of data streams that are within the epoch; and
use only the values that are within the epoch to determine the epochal value.

15. The apparatus of claim 14, wherein the instructions for selecting corresponding epochal values are further executable by the processor to:
use only the most recent values that are within the epoch to determine the epochal value.

16. The apparatus of claim 13, wherein the instructions for selecting corresponding epochal values are further executable by the processor to:
mathematically compute the epochal value from the values that are within the epoch.

17. The apparatus of claim 13, wherein the instructions for selecting corresponding epochal values are executable by the processor to:
identify values from the plurality of values included in the corresponding plurality of data streams that are not within the epoch; and
use the values that are not within the epoch to determine the epochal value.

18. The apparatus of claim 12, wherein the instructions for determining the epochal values are executable by the processor to:
present an option to a user to select, for at least one of the physiological parameters, a physiologically relevant algorithm for determining the corresponding epochal value from among a plurality of algorithms.

19. The apparatus of claim 12, wherein the instructions for selecting the epoch comprise instructions to:
determine, for each of the one or more physiological parameters, a physiologically relevant period of time; and
select a shortest of the determined physiologically relevant periods of time as the epoch.

20. An apparatus for outputting unsynchronized physiological data, comprising:
means for receiving a plurality of data streams each representing a physiological parameter for a person;
means for selecting an epoch;
means for determining, for each of the plurality of data streams, an epochal value to represent each of the physiological parameters for the person in the epoch; and
means for outputting the epochal values with an indication of the selected epoch.
